(19) United States
(12) Patent Application Publication
(10) Pub. No.: US 2003/0171261 A1
(43) Pub. Date: Sep. 11, 2003

(54) Fucosyl gm1-klh conjugate vaccine against small cell lung cancer

(76) Inventors: Philip O Livingston, New York, NY (US); Govindaswami Ragupathi, New York, NY (US)

Correspondence Address: John P White, Cooper & Dunham LLP, 1185 Avenue of the Americas, New York, NY, 10036, US

(21) Appl. No.: 09/534,711

(22) Filed: Mar. 24, 2000

Related U.S. Application Data
(63) Continuation of application No. PCT/US98/20073, filed on Sep. 25, 1998.
Application No. PCT/US98/20073 is a continuation-in-part of application No. 60/059,664, filed on Sep. 25, 1997.

(51) Int. Cl. A61K 38/16; A61K 39/00

(52) U.S. Cl. 514/8; 514/12

(57) ABSTRACT

This invention provides for a composition comprising a fucosyl GM1 ganglioside or an oligosaccharide thereof conjugated to an immunogenic protein, an adjuvant, the amounts thereof being effective to stimulate or enhance antibody production in a subject, and a pharmaceutically acceptable carrier. This invention also provides a method of stimulating antibody production in a subject which comprises administering to the subject an effective amount of the above-described composition to stimulate antibody production. This invention also provides a method of enhancing antibody production in a subject which comprises administering to the subject an effective amount of the above-described composition to enhance antibody production. This invention also provides a method of preventing cancer in a subject which comprises administering to the subject an amount of the above-described composition effective to prevent cancer. This invention also provides a method of treating cancer in a subject which comprises administering to the subject an amount of the above-described composition effective to treat cancer.

This application is claims the benefit of U.S. Provisional Application No. 60/059,664, filed Sep. 25, 1997, the contents of which is hereby incorporated by reference.

The invention disclosed herein was made with Government support under Grant No. PO1CA33049 from the National Institutes Of Health of the United States Department of Health and Human Services. Accordingly, the U.S. Government has certain rights in this invention.

### BACKGROUND

Throughout this application, various publications are referenced by author and date or by Arabic numbers. Full citations for these publications may be found listed alphabetically at the end of the third set of experiments and at the end of the fourth set of experiments immediately preceding the claims. The disclosures of these publications in their entireties are hereby incorporated by reference into this application in order to more fully describe the state of the art as known to those skilled therein as of the date of the invention described and claimed herein.

Lung cancer remains the leading cause of cancer death in the United States, with 160,100 deaths estimated for 1998 (Landis, S. H. et al., 1998). In the United States, lung cancer remains the leading cause of cancer death in men, and has surpassed breast cancer as the leading cause of cancer death in women. Small cell lung cancer (SCLC) accounts for approximately 20% of all lung cancer cases, and is the fifth leading cause of death from cancer (Wingo, P., et al., 1995). Distant metastases are present in more than two-thirds of patients with SCLC at diagnosis (Inhde, D. C., 1995), and in the absence of treatment, tumor progression is rapid, with a median survival of only 2 to 4 months. SCLC, however, is very responsive to chemotherapy, with over 80% of patients with limited stage disease (LD) and >60% of patients with extensive stage disease (ED) achieving a major response to treatment. Despite these results, relapses are common, and most patients die within two years of their diagnosis. For patients who have achieved a major response, after completion of chemotherapy with or without radiation therapy, standard treatment is observation alone. The median survival of patients with LD is 14-20 months, and those with ED is 8-12 months. Over the past decade, no additional therapy has been shown to improve overall survival, and standard therapy is observation alone for patients who have achieved a major response after 4 to 6 cycles of chemotherapy. Because of these modest results, new approaches to adjuvant therapy are needed.

Antibodies produced by B cells are the primary mechanism for the elimination of circulating pathogens from the bloodstream. They can cause rejection of allografts by both acute and chronic mechanisms. Antibodies induce destruction of cells by several mechanisms including opsonification and removal by the reticuloendothelial system, complement mediated lysis, and antibody-dependent cell mediated lysis. They appear ideally suited for eradication of circulating tumor cells and micrometastases in the adjuvant setting (Livingston, P. O., 1995).

Antibodies directed against highly restricted ganglioside antigens present on melanoma cells and a variety of other cancers have been detected in the sera of some patients. It has been noted that the presence of these antibodies has been associated with an unexpectedly favorable course.(Livingston, P. O., 1987; Jones, P. C., et al., 1981). As only few patients have these antibodies in their serum, attempts have been made to induce antibody formation by immunizing patients with tumor vaccines containing relevant antigens.

Adjuvant immunotherapy of SCLC with tumor vaccines must be based on the identification of antigens expressed by SCLC cells which are immunogenic. While several antigens have been identified on SCLC cells using mouse monoclonal antibodies, very few of these are known to be recognized by the human immune system.

Fucosyl-GM1 (Fuc-GM1) is a ganglioside that was initially identified and isolated from bovine thyroid gland (Macher, B. A., et al., 1979). Gangliosides are neuraminic acid containing glycosphingolipids that are anchored into the lipid bilayer of the plasma membrane by their lipophilic ceramide moiety. Specific gangliosides have been found to be specific indicators of carcinomas and may be potential antigenic sites for immunotherapy (U.S. Pat. No. 4,557,931, issued on Dec. 10, 1985).

Gangliosides and most other tumor antigens are poor immunogens because they are autoantigens and are therefore perceived as self. In order to make tumor antigens more immunogenic, they must be taken out of their normal autoantigen environment and placed in the context of immunogenic foreign antigens for presentation to the immune system. Various methods have been used to increase the immunogenicity of antigens, in particular for inducing an IgG response. The approach that has been found to be most successful at inducing an IgG response has been to conjugate gangliosides to immunogenic carrier proteins. GD3, a ganglioside expressed on human malignant melanoma cells, has been covalently attached to keyhole limpet hemocyanin (KLH), derived from a shellfish, in order to improve immunogenicity. High titer IgM and IgG responses against GD3 were seen in mice, which were capable of complement mediated lysis of human melanoma cells expressing GD3 (Helling, F., et al., 1995). However, induction of an immune response or production of antibodies using a vaccine is unpredictable. Though one may induce and produce antibodies in one organism using a particular vaccine, one cannnot predicably state that the same vaccine will induce and/or produce antibodies in another. For example, although low titer IgG and IgM responses were seen in mice against GM2, the same vaccine elicited a high titer production of both IgG and IgM antibodies in patients tested (Livingston, P. O., et al., 1989).

A series of clinical trials have been conducted at Memorial Sloan Kettering Cancer Center (MSKCC) with GM2-KLH conjugate vaccines, and it has been shown that the KLH carrier protein is safe to administer (Helling, F., et al., 1995; U.S. Pat. No. 5,102,663 issued on Apr. 7, 1992). Therefore, KLH will be used as the immunogenic carrier protein in this vaccine.

Groups of melanoma patients have been immunized at MSKCC with melanoma vaccines with no adjuvant or plus various adjuvants: DETOX, BCG and QS-21. QS-21 was a significantly more effective adjuvant than others, producing significantly higher titer IgM and IgG antibodies. QS-21 is a carbohydrate extracted from the bark of the South American tree Quillaja saponaria Molina. The monosaccharide composition, molecular weight, adjuvant effect and toxicity for a series of these saponins have been previously described (Kensil, C. R., et al., 1991). QS-21 was selected due to its adjuvanticity and lack of toxicity. It has also been proven to be nontoxic and highly effective at augmenting the immunogenicity of an FeLV subunit vaccine in cats and an HIV-1 recombinant vaccine in Rhesus monkeys (Newman, M. J., et al., 1992). A Phase I trial demonstrating the safety and adjuvanticity of QS-21 has recently been completed in patients treated with GM2-KLH vaccines (Livingston, P. O., et al., 1994). The 100 μg dose was well tolerated, resulting in erythema and induration at the immunization site lasting 2-3 days and occasional low grade flu-like symptoms, with demonstrated adjuvant activity (International patent application, PCT/US94/00757, filed Jan. 21, 1994 and published under WO 94/16731 on Aug. 4, 1994). Therefore the 100 μg dose has been chosen for this vaccine.

Potential targets for immunotherapy have been identified on the cell surface of SCLC. These include the gangliosides GM2, GD2, GD3, 9-O-acetyl GD3 and Fuc-GM1, as well as the polysialic acid epitope characteristic of the embryonic neural-cell adhesion molecule (N-CAM), the carbohydrate Globo H, and the glycoprotein KSA (Hamilton, W. B., et al, 1993, Zhang, S. et al., 1997, Brezicka, F-T, 1989, Fuentes, R. et al., 1997, Brezicka, F-T, et al. 1992, Cheresh, D. A. et al., 1986, Grant, S. C. et al. 1996, Zhang, S. et al., in press). Of these antigens, the ganglioside Fuc-GM1 is the most restricted in its expression on normal tissues and other types of cancer (Zhang, S. et al., 1997, Brezicka, F-T, 1989). The importance of gangliosides as targets for immunotherapy has been demonstrated by clinical responses observed in melanoma patients after passive immunotherapy with monoclonal antibodies against GM2, GD2, and GD3 (Cheung, N-K., 1987, Houghton, A. N., 1985, Irie, F. R., 1986, Irie, R. F., et al., 1989). In addition, the presence of either naturally occurring antibodies or actively induced antibodies directed against gangliosides has been associated with an improved prognosis (Jones, P. C., et al., 1981 ,Livingston, P. O. et al. 1994, Livingston. P. O., et al. 1989). Previously, SCLC patients have been immunized after initial chemotherapy with BEC2, an anti-idiotypic monoclonal antibody that mimics GD3 (Grant, S. C., et al. 1996). Patients developed anti-GD3 antibodies and had prolonged survival compared to historical controls. With these encouraging results, we are investigating Fucosyl-GM1 as a target for immunotherapy.

Fuc-GM1 was initially identified and isolated from the bovine thyroid gland (Macher, B. A., et al. 1979). With the use of a highly specific mouse monoclonal antibody, F12, the ganglioside Fuc-GM1 (Fucα1-2Galβ1-3GalNAcβ1-4 (NeuAcα2-3)Galβ1-4Glcβ1-1Cer) was identified in the majority of SCLC tissue samples and the serum of a few patients with the disease (Zhang, S., et al., 1997, Brezicka, F-T., et al 1989, Fredman, P., et al., 1986, Vangsted, A. J., et al. 1991.). Fuc-GM1 was not detected in normal lung and bronchus, but was sparsely distributed in occasional small round cells in the thymus, spleen, pancreatic islet cells, lamina propria and intramural ganglionic cells of the small intestine, as well as a small subset of peripheral sensory neurons and dorsal root ganglia (Zhang, S., et al., 1997, Brezicka, F-T., et al 1989, Yoshino, H., et al., 1993).

Serum antibodies against Fuc-GM1 have been described in a few patients with sensory neuropathies but not in other settings, suggesting that this antigen is poorly immunogenic. We have explored a variety of approaches for augmenting the immunogenicity of poorly immunogenic antigens. The most effective of these methods has been chemical conjugation to keyhole limpet hemocyanin (KLH), a shellfish-derived protein, followed by mixture with the immunological adjuvant QS-21 (Livingston, P. O., et al., 1987, Helling, F., et al., 1994, Helling, F., et al., 1995, Livingston, P. O., et al., 1994, Kensil, C. R.., et al. 1991). In this study, ten patients with SCLC achieving a major response to standard therapy have received at least 5 vaccinations with a Fucosyl-GM1-KLH conjugate vaccine, and reactivity of the induced antibody response has been evaluated.

### SUMMARY OF THE INVENTION

This invention provides composition comprising a fucosyl GM1 ganglioside or a oligosaccharide portion thereof conjugated to an immunogenic protein, an adjuvant, the amounts thereof being effective to stimulate or enhance antibody production in a subject, and a pharmaceutically acceptable carrier.

This invention also provides a method of stimulating antibody production in a subject which comprises administering to the subject an effective amount of the above-described composition to stimulate antibody production.

This invention further provides a method of enhancing antibody production in a subject which comprises administering to the subject an effective amount of the above-described composition to enhance antibody production.

This invention further provides a method of preventing cancer in a subject which comprises administering to the subject an amount of the above-described composition effective to prevent cancer.

This invention further provides a method of treating cancer in a subject which comprises administering to the subject an amount of the above-described composition effective to treat cancer.

### BRIEF DESCRIPTION OF FIGURES

FIG. 1. Expression of carbohydrate antigens on melanoma and small cell lung cancer. SCLC cells were strongly stained with mAb F12 against fucosyl GM1.

FIG. 2. Expression of carbohydrate antigens on normal tissues. Luminal cells of normal breast were moderately stained (2-3+) with MAb 696 against GM2(a) and MAb MBr1 against Globo H (b), Luminal cells of colon mucosa were strongly stained (3+) with MAb 696 against GM2(c) Cells in white pulp of spleen but not in red pulp were strongly stained (3-4+) with MAb 3F8 against GD2(d). Strong immunnostaining (3-4+) was detected on gray matter of brain and moderate (1-2+) staining on white matter with MAb 3F8 against GD2(e). Scale bar 100 μm.

### DETAILED DESCRIPTION OF THE INVENTION

Abbreviations:

The designations GD3, GD2, GM2, 9-O-acetyl-GD3 and fucosyl GM1 are used in accordance with the abbreviated ganglioside nomenclature proposed by Svennerholm (1963). ABC, avidin-biotin complex; ITLC, immune thin layer chromatography; mAb, monoclonal antibody; PBS, phosphate buffered saline; SCLC, small cell lung cancer; MSKCC, Memorial Sloan-Kettering Cancer Center.

This invention provides a composition comprising a fucosyl GM1 ganglioside or a oligosaccharide portion thereof conjugated to an immunogenic protein, an adjuvant, the amounts thereof being effective to stimulate or enhance antibody production in a subject, and a pharmaceutically acceptable carrier.

The oligosaccharide portion of fucosyl GM1 ganglioside may be derived by cleaving the ganglioside or it may be synthesized directly.

In a specific embodiment, the amount of the fucosyl GM1 is an amount between about 3 μg to about 100 μg.

As used herein, an immunogenic protein is a protein or derivative thereof that, when conjugated to the ganglioside or oligosaccharide portion thereof, stimulates or enhances antibody production in the subject. Keyhole Limpet Hemocyanin is a well-known immunogenic protein. A derivative of Keyhole Limpet Hemocyanin may be generated by direct linkage of at least one immunological adjuvant such as monophospholipid A or non-ionic block copolymers or cytokine with Keyhole Limpet Hemocyanin. Cytokines are well known to an ordinary skilled practitioner. Example of cytokine are granulocyte macrophage colony stimulating factor (GMCSF) and interleukin 2. There are other known interleukins in the art which may be linked to Keyhole Limpet Hemocyanin, forming a derivative of Keyhole Limpet Hemocyanin.

In a specific embodiment, the composition comprises fucosyl GM1 ganglioside conjugated to Keyhole Limpet Hemocyanin or a derivative thereof conjugated to the ganglioside through the ceramide portion thereof, specifically, the ganglioside is conjugated to Keyhole Limpet Hemocyanin.

In a further embodiment, the adjuvant is a carbohydrate derived from the bark of a Quillaja saponaria Molina tree, specifically QS-21, and is an amount between about 30 μg to about 100 μg. There are other known adjuvants which may be applicable to this invention. There may be classes of QS-21 or QS-21 like chemicals which may be similarly used in accordance with this invention.

Different effective amounts of the conjugated ganglioside or oligosaccharide portion thereof, and the adjuvant may be used according to this invention. A person of ordinary skill in the art can perform simple titration experiments to determine the effective amounts required for effective immunization. An example of such titration experiment is to inject different amounts of the conjugated ganglioside or conjugated oligosaccharide portion thereof or adjuvant to the subject and then examine the immune response.

For the purposes of this invention “pharmaceutically acceptable carrier” means any of the standard pharmaceutical carrier. Examples of suitable vehicles are well known in the art and may include, but not limited to, any of the standard pharmaceutical vehicles such as a phosphate buffered saline solutions, phosphate buffered saline containing Polysorb 80, water, emulsions such as oil/water emulsion, and various type of wetting agents.

The vaccine of this invention may be administered intradermally, subcutaneously and intramuscularly. Other methods well known by a person of ordinary skill in the art may also be used.

In specific embodiment of the invention, the subject is a human being.

This invention also provides a method of stimulating antibody production in a subject which comprises administering to the subject an effective amount of the above-described composition to stimulate antibody production. In a specific embodiment, the composition comprises fucosyl GM1 ganglioside conjugated to Keyhole Limpet Hemocyanin or a derivative thereof conjugated to the ganglioside through the ceramide portion thereof; even more specifically, the ganglioside is conjugated by its ceramide portion to Keyhole Limpet Hemocyanin.

This invention also provides a method of enhancing antibody production in a subject which comprises administering to the subject an effective amount of the above-described composition to enhance antibody production.

This invention also provides a method of preventing cancer in a subject which comprises administering to the subject an amount of the above-described composition effective to prevent cancer, specifically, the cancer is small cell lung cancer.

This invention also provides a method of treating cancer in a subject which comprises administering to the subject an amount of the above-described composition effective to treat cancer, specifically, the cancer is small cell lung cancer.

This invention is illustrated in the Experimental Details section which follows. These sections are set forth to aid in an understanding of the invention but are not intended to, and should not be construed to, limit in any way the invention as set forth in the claims which follow thereafter.

### EXPERIMENTAL DETAILS

Understanding the distribution of tumor associated antigens on cancers and normal tissues is essential for selection of targets for cancer immunotherapy. Seven carbohydrate antigens, potential targets for immunotherapy, were studied using a panel of well characterized monoclonal antibodies by immunohistochemistry on cryostat-cut tissue sections of 13 types of cancers and 18 normal tissues. Fucosyl GM1 was detected only on small cell lung cancers (SCLC). Fucosyl GM1 was not expressed significantly on any of the normal tissues analyzed. This study extends understanding of the distribution of the carbohydrate antigen fucosyl GM1 and provides a more solid basis for selection of appropriate carbohydrate antigens for immune attack, the optimal tumor targets and the normal tissues susceptible to injury in the process.

Introduction

Carbohydrate antigens are the most abundantly expressed antigens on the cell surface of most cancers (Hakomori et al., 1985; Feizi et al., 1985; Livingston et al., 1992; Hamilton et al., 1993a,b). Several carbohydrate antigens, such as gangliosides GD3, GD2, GM2 and the disaccharide sTn, have been shown to function as effective targets for passive immunotherapy with monoclonal antibodies (Houghton et al., 1985; Cheung et al., 1987; Saleh et al., 1992; Irie et al., 1986; Schlom et al., 1992). They have also been demonstrated to be effective targets for active immunotherapy with vaccines in clinical trials (Livingston et al., 1994; MacLean et al., 1993). An important step in selection of carbohydrate antigens as candidates for targets in immunotherapy trials is determining their distribution in malignant and normal tissues. The availability of monoclonal antibodies (mAb) against these antigens for investigating the antigen expression in tissue sections by immunohistochemistry has facilitated these studies (Feizi et al., 1985).

Immunohistology is notoriously inconsistent for quantitating antigen expression, especially when results from different laboratories are compared. It has been difficult to select optimal antigens and tumor targets based on these previous studies. The distribution of the antigens studied here has been described (Dippold et al., 1985; Bernhard et al., 1992; Cheresh et al., 1984; Brezicka et al., 1989; Bremer et al., 1984; Husmann et al., 1990), but number and types of tissues studied were generally limited and involved mAbs against one or two antigens without comparison with the expression of other antigens. For this purpose, a large immunohistochemical study on frozen tissue sections of tumor and normal tissues using a panel of well characterized murine mAbs against the antigen fucosyl GM1 was begun. Described here is the distribution of the ganglioside fucosyl GM1. Gangliosides such as GM3, GM1 and GD1a were not considered potential targets for immunotherapy due to their known extensive expression on normal tissues and so were not tested here. The study that follows describes the expression of blood group-related antigens on this same panel of tissues.

### 1. First Set of Experiments

Materials and Methods

A. Tissue Sample

Frozen specimens embedded in Tissue-Tek O.C.T. compound (Diagnostic Division, Elkhart, Ind.) were provided with pathological reports by the Tissue Procurement Service of Memorial Sloan-Kettering Cancer Center (MSKCC). Cryostat sections were cut at 5-6 μm, dried in air and fixed with neutral buffered 10% formalin solution (Sigma Co, St. Louis, Mo.) or methanol (Fisher Scientific, Fair Lawn, N.J.) for 10 min before hematoxylin-eosin or immune staining.

B. mAb and Immunohistochemistry

mAB F12 (antigen: fucosyl GM1 (Fucα1→2Galβ1→3GalNAcβ1→4(NeuAc2→3)Galβ1→4Glcβ1→1Cer was provided by Dr. Thomas Brezicka (Goteborg University, Sweden).

The avidin-biotin complex (ABC) immunoperoxidase method was performed as previously described (Hsu et al., 1981). Briefly, the sections were quenched with 0.1% H₂O₂ in PBS for 15 min, blocked with avidin and biotin reagents (Vector Laboratories, Inc. Burlingame, Calif.) for 10 min each, incubated in 10% serum of horse or goat from which the second antibody was raised, and incubated with various mAbs for 1 h at optimal concentration. The optimal mAb concentration was selected based on strong reactivity against the known positive target cells and little or no background against stroma. The concentration of mAb used was F12 at 1.5 μg/ml. D1.1 is a supernatant and was used at 1:4. The sections were subsequently incubated with 1:600 biotinylated horse anti-mouse IgG or 1:300 goat anti-mouse IgM antibodies (Vector Laboratories, Inc. Burlingame, CA ) for 40 min, and then incubated in 1:50 ABC reagent (Vector Laboratories, Inc. Burlingame, Calif. ) for 30 min. Reactions were developed with 0.02% H₂O₂ and 0.1% diaminobenzidine tetrahydrochloride (Sigma Co., St. Louis, Mo. ) for 2-5 min. Slides were then counterstained with Harris modified hematoxylin (Fisher Scientific, Fair Lawn, N.J.) for 1-3 min. The immunoreactivities were graded based on the percentage of positive cells and staining intensity above that seen on the negative control: 1+ (weak), 2+ (moderate), 3+ (strong) and 4+ (very strong). Known positive and negative control slides were used in each experiment. Results with the several IgM, IgG3 and IgG2 mAbs included in the panel of antibodies tested ruled out non-specific adherence of particular subclasses of antibodies.

An indirect immunoperoxidase method was performed, as previously described (Cheresh et al., 1984), on normal liver, kidney and stomach samples. These tissues reacted strongly with ABC reagent directly, producing high background. Briefly, the sections were quenched with 0.1% H₂O₂ in PBS for 15 min, blocked with 10% serum and incubated with mAbs for 1 h at the optimal concentration which for this assay was F12 at 20 μg/ml, and D1.1 at the full strength supernatant. The sections were incubated with 1:100 rabbit anti-mouse immunoglobulin labeled with peroxidase (Dako Co., Cappinteria, Calif.) for 1 h and developed as described for the ABC method.

C. Immune Thin-Layer Chromatography (ITLC)

Extraction of acidic and neutral glycolipids from tissues and ITLC were performed as previously described (Hamilton et al., 1993a). Two to five μg of extracted glycolipids, and GM2 and GD2 controls, were loaded on high performance silica gel plates (Merck Co, Darmstadt, Germany) and separated in chloroform/methanol/0.02% aqueous CaCl2 (60:35:8; v/v/v) running solvent. One plate was stained for visualizing the whole glycolipids with resorcinol or orcinol. The other one was incubated with mAb 696 (5 μg/ml), and then with rabbit anti-mouse IgM conjugated with horseradish peroxidase (Zymed Co., San Francisco, Calif.) and developed in 4-chloro-1-napthol solution (Sigma Co., St Louis, Mo.) containing H₂O₂ (Fisher Co., Fair Lawn, N.J.).

Results

A. Reactivity of the mAbs with Tumor Tissues

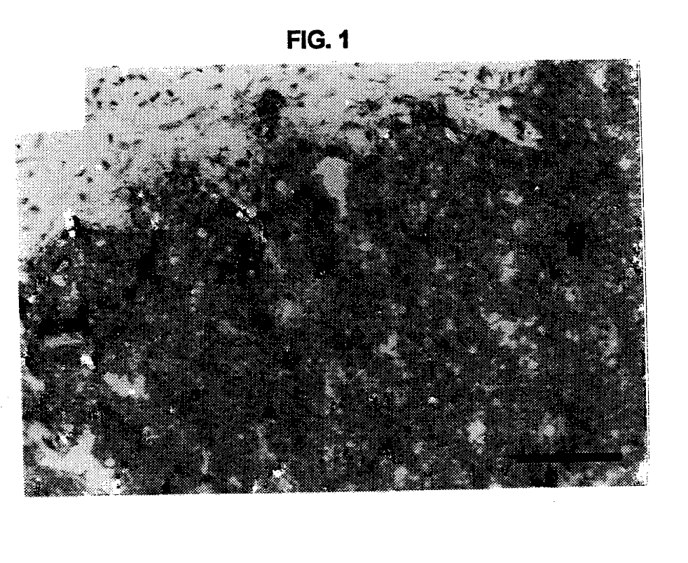

Table 2 summaries the immunoreactivities on tumor tissue samples observed with the panel of mAbs. Overall 73 neoplastic tissues were analyzed with each of the 8 antibodies. Examples of results on melanoma and small cell lung carcinoma are shown in FIG. 1. Fucosyl GM1 had highly restricted distributions: SCLC alone.

B. Reactivity of the mAbs with Normal Tissues

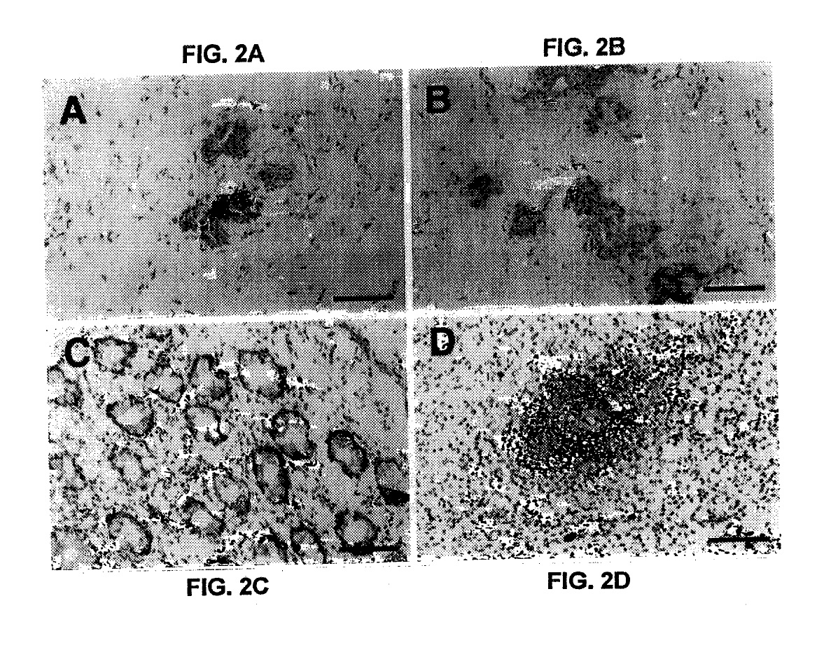

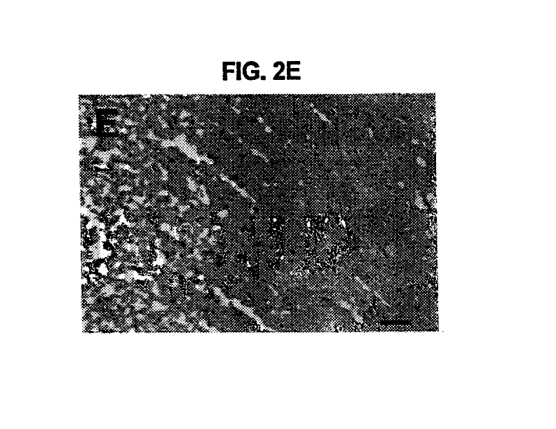

Table 3 summarizes the immunoreactivities on normal tissue samples observed with the panel of mAbs. Examples of results on normal tissues are shown in FIG. 2. mAb F12 only reacted with occasional pancreatic islets of Langerhans. cells (less than 10% of islet cells) and occasional dorsal root ganglion neurons.

Discussion

An unexpected finding in the study was the remarkably restricted distribution of fucosyl GM1. Fucosyl GM1 was expressed on SCLC, as previously reported (Brezicka et al., 1989). Fucosyl GM1 was not found in any other cancers or normal tissue tested except for weak staining on fewer than 10% of cells in the islets of Langerhans, and occasional dorsal root ganglion neurons. This is a more limited distribution in the islets and other tissues than previously observed by Brezicka et al (1989) using immunofluorescence. Fucosyl GM1 appears to be an excellent target for immune attack against small cell lung cancer.

There is accumulating evidence that it is not the overall quantity of a given antigen on normal tissues which is the primary determinant of its usefulness as a target for active and passive immunotherapy of cancer but the precise distribution of where the antigen is overexpressed and its availability to the immune system. Gangliosides GD2 and GD3 are widely distributed in the central nervous system (CNS) and at lower levels in the stroma of most organs, but passive treatment of children and adults with moderate doses of mAbs against GD2 and GD3 has resulted in clinical responses in the absence of CNS symptoms or autoimmunity. The blood brain barrier appears to prevent access of these antibodies to the CNS. The known high expression of GD2 on peripheral nerves, however, has resulted in dose dependent acute and/or chronic toxicity in some patients treated with high doses of mAbs against GD2 (Saleh et al., 1992). Since the distribution of GD2 on B cells has not previously been suspected, no attempt at evaluating B cell number or function was made in these trials. The distribution of GM2 on normal tissues is shown here to be much more widespread than GD2 or GD3 but it may be more restricted to sites which are not accessible to the immune system, the brain (though less than GD2 and GD3) and the epithelial luminal cells of most organs. No expression of GM2 in stroma or connective tissue elements was detected. Antibodies against GM2 and several other antigens with this type of distribution in epithelial tissues such as MUC1, sTn and TF have been induced or administered without evidence of toxicity or autoimmunity (MacLean et al., 1992, 1993; Finn et al., 1995; Gilewski et al., 1996; Adluri et al., 1995). Induction of antibodies against GM2 following immunization with properly constructed vaccines is seen in most patients and has been associated with a significantly better prognosis, again in the absence of any evidence of autoimmunity (Livingston., 1994). It appears that this distribution on normal tissues neither induces tolerance nor is available to antibodies once induced. Against this background, fucosyl GM1 appears to be an outstanding target for passive or active immunotherapy.

### 2. Second Set of Experiments

The use of a vaccine containing fucosyl GM1 covalently conjugated to Keyhole Limpet Hemocyanin (fucosyl GM1-KLH) plus the immunological adjuvant QS-21 in patients with small cell lung cancer will be observed. Similarly to a study in which more than 90 patients were immunized with GM2-KLH plus QS-21 were immunized (Livingston, P. O., et al., Helling, F., et al, 1995), such an immunization study will be performed using the fucosyl GM1-KLH vaccine. Fucosyl-GM1 (Fuc-GM1) is a ganglioside that was initially identified and isolated from bovine thyroid gland (Macher, B. A., et al., 1979). Gangliosides are neuraminic acid containing glycosphingolipids that are anchored into the lipid bilayer of the plasma membrane by their lipophilic ceramide moiety. With the use of a highly specific mouse monoclonal antibody, F12, (Fredman, P., et al., 1986) the ganglioside, Fuc-GM1 (Fucα1-2Galβ1-3GalNAcβ1-4(NeuAcα2-3)-Galβ1-4Glcβ1-1Cer) was identified in tissue samples of nineteen of 21 cases of SCLC and was also detected in serum of a few patients with the disease. Fuc-GM1 was not identified in normal lung and bronchus, however sparsely distributed clusters of small round cells were stained in the thymus, spleen, pancreatic islet cells, and the lamina propia and intramural ganglionic cells of the small intestine (Brezicka, F-T., et al., 1989; Vangsted, A. J., et al., 1991; Yoshino, H., et al., 1993).

Fucosyl GM1 conjugated to Keyhole Limpet Hemocyanin and adjuvant has not been used previously to immunize patients. Fucosyl GM1 was initially identified as a cancer antigen using murine monoclonal antibodies including mAB F12 (Fredman, P., et al., 1986; Nilsson, O, et al., 1986; Brezicka, F-T., et al., 1989). The distribution of fucosyl GM1 on normal tissues is sufficiently restricted and the distribution on small cell lung cancer is sufficiently generalized to suggest that fucosyl GM1 would be an excellent target for immunotherapy. It would be possible to actively immunize against fucosyl GM1 using a fucosyl GM1-KLH conjugate vaccine plus the immunological adjuvant QS-21.

Preclinical Studies with Fucosyl GM1-KLH Plus QS-21 Vaccines:

Using fucosyl GM1 prepared by Matreya Inc., the immunogenicity of several different fucosyl GM1 vaccines was compared. Fucosyl GM1 conjugated to KLH plus the immunological adjuvant QS-21 was the most immunogenic approach. IgM and IgG titers detected by ELISA were highest with this approach, especially when the final vaccine was lyophilized during vialing instead of storing it in saline at 4° C . These results are summarized Table 1. They demonstrate that the fucosyl GM1-KLH plus QS-21 vaccine is immunogenic in mice, inducing high titer antibodies against Fuc GM1.

TABLE 1
Mice Immune Response To The Fucosyl GM1-KLH Vaccine

| | ELISA TITER | |
|---|---|---|
| | (MEDIAN OF 5 MICE) | |
| | IgM | IgG |
| Fuc GM1 | 0 | 0 |
| Fuc GM1 + KLH + QS-21 | 1/300 | 0 |
| Fuc GM1-KLH | 1/300 | 0 |
| Fuc GM1-KLH + QS-21 stored at 4° C. | 1/900 | 1/300 |
| Fuc GM1-KLH + QS-21 lyophilized | 1/24,000 | 1/900 |

1. Immunization Using Fucosyl GM1-KLH Conjugate Plus the Immunological Adjuvant QS-21 in Patients with Small Cell Lung Cancer who Have Achieved a Major Response to Initial Therapy

Fucosyl GM1 is further purified and covalently attached to KLH.

2. Chemistry, Manufacturing and Control Data

A. Fucosyl GM1 Extraction

Bovine thyroid glands obtained from domestic cows were extracted according to the method described by Van Dessel et al, a method very similar to the one used in preparation of GM2 and GD2 from bovine brain for many previous trials with ganglioside vaccines. In brief, thyroid tissue was lyophilized and extracted by the Folch system with varying concentrations of chloroform and methanol. Non-lipid contaminants were removed by Sephadex G-25 chromatography and fucosyl GM1 separated by preparative thin layer chromatography (TLC). The fucosyl GM1 is received from Matreya Inc. in chloroform/methanol (2/1), evaporated and reconstituted in methanol. Purity is tested by TLC and ITLC. In the past there has been no evidence of any contamination, only the single fucosyl GM1 band was present (>95% pure).

B. Conjugation of Fucosyl GM1 to KLH:

Keyhole limpet hemocyanin (KLH) is purchased from Perimmune Inc. and used under the Perimmmune Inc. IND (BB-IND 4250). Extensive experience exists in the clinic with this KLH in GM2-KLH, GD2-KLH, globo H-KLH and MUC-KLH conjugate vaccines prepared in the laboratories. Conjugation of fucosyl GM1 to KLH will be performed in the laboratory of Dr. Livingston using exactly the same methods that were used previously for conjugating gangliosides to KLH.

C. Synthesis of Fucosyl GM1 Aldehyde:

All glassware is rinsed with distilled water and autoclaved prior to use. A solution of purified fucosyl GM1 (5 mg) in methanol (5 ml) is stirred at room temperature and ozone gas (Delzone Traveler Model Z0-150 ozonator) is passed through the solution for 10 min. A stream of nitrogen is passed through the solution to remove excess ozone and the reaction is checked by TLC in chloroform/methanol/water (60/35/8). To this solution is added methylsulfide (400 ul) and the reaction mixture is stirred at room temperature for 2 hours. The solvents are removed under a stream of nitrogen and treated with n-hexane to remove free fatty aldehydes. The resulting white powder is used directly in the subsequent conjugation step.

D. Conjugation of Fucosyl GM1 to KLH:

All manipulations are performed in a Class 100 biological safety cabinet. The aldehyde from 7.21 is dissolved in sterile, pyrogen-free PBS (pH 7.5) in a flask under sonication, then transferred to a sterile glass bottle containing 10 mg of sterile, pyrogen-free KLH dissolved in PBS (5 mg/ml) (Perimmune Inc., Rockville, Md.). The flask is rinsed two more times with 2.5 ml of PBS, which is added to the KLH/fucosyl GM1 aldehyde mixture and allowed to incubate at room temperature for 15 minutes with gentle stirring.

Two ml of a 20 mg/ml solution of sodium cyanoborohydride (NaBH₃CN) is prepared in PES and sterile filtered. A 1 ml volume of the NaBH₃CN solution is added to the KLH/fucosyl GM1 aldehyde mixture and incubated at 37° C. for 48 hr.

E. Diafiltration of Fucosyl GM1-KLH Glycoconjugate:

All manipulations are performed in a Class 100 biological safety cabinet. The contents of the fucosyl GM1-KLH reaction vials are transferred to sterile, pyrogen-free Amicon Centriprep concentrator 30 units and centrifuged at 1500 g for 15 min. The conjugates are then washed 3 times with the same procedure using saline (injection USP) and are aseptically removed from the filtration unit and spun at 2000 rpm for 30 min. The supernatants are then sterile filtered with a 0.22 μm low protein binding sterile, pyrogen-free filter and stored at −20° C. Protein and ganglioside content is determined and the solution is suitably diluted in saline. QS-21 is added to yield 100 ug/ml and the mixture is 0.22 .Am filtered again and aliquoted in 1 ml to sterile 2 ml nunc vials. The vials are lyophilized, capped and stored at −30° C.

F. Lot Release Criteria:

Fucosyl GM1 must be at least 95% pure by TLC. Fucosyl GM1-KLH ratios between 400/1 and 1400/1 assuming a KLH molecular weight of 5×10⁶ will be accepted. TLC or ITLC will be performed with each lot of fucosyl GM1-KLH for determination of percent unbound fucosyl GM1 and for comparison to future lots. No more than 20% unbound fucosyl GM1 is acceptable. Sterility and safety testing with vials from each lot at >50 times the dose/meter² to be used in clinical trials will be performed. No growth in culture and no adverse reaction in mice or guinea pigs (including weight loss of 10% or more) will be tolerated. Two or more mice will be immunized with each fucosyl GM1-KLH batch on 2-3 occasions at 1-2 week intervals and post immunization sera tested. Antibody titers of 1/1000 or greater against fucosyl GM1 will be accepted as proof that the construct has the appropriate immunogenicity.

3. Pharmacology and Toxicology Data

The expression of fucosyl GM1 on normal tissues in the mouse or rabbit has not been studied but no evidence of toxicity in the mice immunized in the course of studies with fucosyl GM1-KLH plus QS-21 vaccines was seen. Given the restricted distribution of fucosyl GM1 on normal tissues, and our long experience with KLH conjugate vaccines plus immunological adjuvant QS-21 in patients, it is considered unlikely that unexpected toxicity will result from this vaccine.

However, fucosyl GM1 is present in occasional cells of the thymus, spleen, pancreatic islet cells, lamina propia and intramural ganglionic cells of. the small intestine, peripheral sensory neurons, and possibly other normal tissues. Several patients with peripheral sensory neuropathy have had antibodies in their serum against GM1 and fucosyl-GM1, raising the possibility that peripheral sensory neuropathy could develop after vaccination (21). These locations are in a more restricted distribution on normal cells than other antigens used as targets for immunotherapy including GM2, GD2, GD3 and epidermal growth factor receptor. If a cross reaction does occur, then autoimmunity against these tissues may result in diabetes mellitus, inflammatory bowel disease, pancreatitis or peripheral sensory neuropathy. It is felt that such inflammatory reactions would be controlled by cessation of vaccination and anti-inflammatory medications. All patients will be evaluated prior to the first vaccination with a baseline history and physical, including a neurological examination, as well as a follow up evaluation before the fifth and sixth vaccination. These vaccinations will not be administered if there is evidence of development of peripheral neuropathy, or diabetes mellitus, pancreatitis or gastrointestinal disease felt to be secondary to the vaccinations.

### 3. Third Set of Experiments

Using the procedures described hereinabove, applicants vaccinated eight individuals with fucosyl GM1-KLH vaccine having as an adjuvant, QS-21. All patients had a diagnosis of small cell lung cancer and had received chemotherapy and radiation therapy, the standard therapy for this disease. All vaccines contained 30 μg Fuc GM1 conjugated to KLH and 100 μg QS-21 and were administered at weeks 1, 2, 3, 4, 8 and 16 subcutaneously.

After each vaccination, serum samples from the eight individuals were collected. And the characteristics of antibody populations were analyzed. Using an enzyme-linked immunosorbent sandwich assay the titers of IgM and IgG in the serum samples were determined (Table 4).

The results shown in Table 4 of the vaccinations in these patients using fucosyl GM1-KLH in combination with the QS-21 adjuvant clearly show induction of an immune response. The immune response induced by the fucosyl GM1-KLH vaccine not only produced IgM antibodies, but as well, IgG antibodies. Such a vaccine could then be used to enhance or produce antibodies against the fucosyl GM1 antigen found on carcinomas, such as small cell lung cancer. It should be noted that the only toxicity seen was local erythemas and induration at injection sites and low grade fever for one to two days, the expected side effects from QS-21. The patients remain well and will be followed for possible delayed toxicity or protection from disease recurrence, which is expected in over 80% of these patients.

TABLE 2
Number Of Tumor Specimens With 50% Or More Of Cells Positive By Immunohistology

| | Antigen (mAb) | | | | | | | |
|---|---|---|---|---|---|---|---|---|
| | | | | | | | Poly | Poly |
| | | 9-O—Ac- | | | Fucosyl | | Sialic | Sialic |
| | GD3 | GD3 | GD2 | GM2 | GM1 | Globo H | Acid | Acid |
| Tumor | (R24) | (D1.1) | (3F8) | (696) | (F12) | (Mbr1) | (735) | (NP-4) |
| Melanoma | 8/10 | 4/10 | 6/10 | 10/10 | 0/10 | 0/10 | 0/10 | 0/5 |
| Sarcoma | 4/9 | 0/9 | 5/9 | 8/9 | 0/9 | 0/9 | 1/9 | 0/5 |
| Neuroblastoma | 4/5 | 0/5 | 5/5 | 5/5 | 0/5 | 0/5 | 5/5 | 1/5 |
| Small Cell Lung | 0/6 | 0/6 | 0/6 | 6/6 | 4/6 | 4/6 | 6/6 | 5/6 |
| Breast | 0/5 | 1/5 | 0/5 | 5/5 | 0/5 | 4/5 | 0/5 | 0/5 |
| Prostate | 0/5 | 0/5 | 0/5 | 5/5 | 0/5 | 2/5 | 0/5 | 0/5 |
| B Cell Lymphoma | 0/5 | 0/5 | 4/5 | 3/5 | 0/5 | 0/5 | 0/5 | 0/5 |
| Lung | 0/5 | 0/5 | 0/5 | 4/5 | 0/5 | 3/5 | 0/5 | 0/5 |
| Colon | 0/5 | 0/5 | 0/5 | 5/5 | 0/5 | 0/5 | 0/5 | 0/5 |
| Pamcreas | 0/5 | 0/5 | 0/5 | 5/5 | 0/5 | 5/5 | 0/5 | 2/5 |
| Gastric | 0/5 | 0/5 | 0/5 | 4/5 | 0/5 | 4/5 | 0/5 | 1/5 |
| Ovarian | 0/5 | 0/5 | 0/5 | 5/5 | 0/5 | 3/5 | 0/5 | 0/5 |
| Endometrial | 0/5 | 0/5 | 0/5 | 5/5 | 0/5 | 4/5 | 1/5 | 1/5 |

^{a}All the tumor tissues were stained by Avidin-Biotin Complex immunoperoxidase methods.

TABLE III
ANTIGEN EXPRESSION ON NORMAL TISSUES DEFINED BY IMMUNOHISTOLOGY¹

| | Antigen (MAb) | | | | | | | |
|---|---|---|---|---|---|---|---|---|
| Normal | GD3 | 9-O-Acetyl | GD2 | GM2 | Fucosyl | Poly sialic | Poly sialic | Globo II |
| (number) | (R24) | (D1.1) | (3F8) | (696) | GM1 (F12) | acid (735) | acid (NP-4) | (Mbr1) |
| Brain (3) | | | | | | | | |
| Gray matter² | 2+ | − | 4+ | 2+ | − | 3+ | − | − |
| White matter | 1+ | − | 2+ | − | − | − | − | − |
| Spleen (3) | | | | | | | | |
| White pulp | − | − | 3+ | − | − | − | − | − |
| Red pulp | − | − | − | − | − | − | − | − |
| Lymph node (2) | −³ | − | 1+⁴ | +/− | − | −³ | − | − |
| Striated muscle (3) | − | − | − | − | − | − | − | − |
| Smooth muscle | | | | | | | | |
| Colon (2) | − | − | − | − | − | − | − | − |
| Blood vessel (3) | − | − | − | − | − | − | − | − |
| Stomach (2) | − | − | − | − | − | − | − | − |
| Uterus (3) | − | − | 3+ | − | − | − | − | − |
| Epithelia | | | | | | | | |
| Lung (2) | − | − | − | 3+⁵ | − | 3+⁵ | 3+⁵ | 3+ |
| Breast (3) | − | − | − | 3+ | − | − | − | 3+ |
| Prostate (2) | − | − | − | 4+ | − | − | − | 2+ |
| Colon (2) | − | − | − | 4+ | − | − | 2+ | − |
| Stomach (2) | − | − | − | 3+ | − | − | 2+ | 3+ |
| Pancreas (2) | − | − | − | 3+ | ⁶ | − | 2+ | 4′ |
| Uterus (1) | − | − | − | 3+ | − | − | − | 4+ |
| Ovary (2) | − | − | − | 3+ | − | − | − | 4+ |
| Liver (2) | − | − | − | − | − | − | − | − |
| Kidney (2) | − | − | − | 2+ | − | − | − | − |
| Connective tissues | | | | | | | | |
| Lung (2) | − | − | − | − | − | − | − | − |
| Breast (3) | 2+ | − | 3+ | − | − | − | − | − |
| Prostate (2) | 2+ | 2+ | 2+ | 1+ | − | − | − | − |
| Colon (2) | − | − | 3+ | − | − | 3+⁷ | − | − |
| Stomach (2) | 2+ | 2+ | 2+ | − | − | − | − | − |
| Pancreas (2) | − | − | − | − | − | − | − | − |
| Uterus (3) | 2+ | − | 2+ | − | − | − | − | − |
| Ovary (2) | 2+ | 2+ | 3+ | − | − | − | − | − |
| Liver (2) | − | − | − | − | − | − | − | − |
| Kidney (2) | 1+ | − | 1+ | − | − | − | − | − |
| ¹All the tissues were stained by Avidin-Biotin Complex immunoperoxidase method except stomach, liver and kidney, which were stained by indirect immunoperoxidase method. −²Only neurons in gray matter were stained. −³A few macrophage like cells (less than 5%) were stained. −⁴Only germinal centers were stained. −⁵In addition to bronchial epithelia 3+, pneumocytes in alveolar spaces 2+. | | | | | | | | |
| # −⁶A few islet cells (less than 10%) in the pancreas were stained 1-2+. −⁷Capillary endothelial cells and ganglion neurons in plexus 3+. | | | | | | | | |

TABLE 4
Study of Human Immune Responses to Fucosyl-GM1-
KLH + QS-21 Vaccination

| Patient | Vaccination | | | ELISA | ELISA |
|---|---|---|---|---|---|
| No. | Dates | Sera Dates | Sera # | (IgM) | (IgG) |
| 1 | May 30, 1997 | May 30, 1997 | FG1 | 20 | 0 |
| | Jun. 5, 1997 | Jun. 5, 1997 | FG2 | 10 | 0 |
| | Jun. 13, 1997 | Jun. 13, 1997 | FG3 | 20 | 0 |
| | Jun. 19, 1997 | Jun. 19, 1997 | FG4 | 160 | 320 |
| | | Jul. 7, 1997 | FG7 | 320 | 320 |
| | Jul. 17, 1997 | Jul. 17, 1997 | FG11 | 160 | 160 |
| | | Jul. 31, 1997 | FG15 | 160 | 160 |
| 2 | Jun. 26, 1997 | Jun. 26, 1997 | FG5 | 0 | 0 |
| | Jul. 3, 1997 | Jul. 3, 1997 | FG6 | 0 | 0 |
| | Jul. 10, 1997 | Jul. 10, 1997 | FG8 | 0 | 0 |
| | Jul. 17, 1997 | Jul. 17, 1997 | FG10 | 160 | 20 |
| | | Jul. 30, 1997 | FG26 | 160 | 160 |
| | Aug. 14, 1997 | Aug. 14, 1997 | FG20 | 160 | 40 |
| 3 | Jul. 15, 1997 | Jul. 15, 1997 | FG9 | 0 | 0 |
| | Jul. 22, 1997 | Jul. 22, 1997 | FG12 | 0 | 0 |
| | Jul. 29, 1997 | Jul. 29, 1997 | FG14 | 10 | 40 |
| | Aug. 5, 1997 | Aug. 5, 1997 | FG17 | 160 | 640 |
| | | Aug. 19, 1997 | FG22 | 160 | 1280+ |
| | Sep. 2, 1997 | Sep. 2, 1997 | FG32 | 320 | 640 |
| 4 | Jul. 25, 1997 | Jul. 25, 1997 | FG13 | 0 | 10 |
| | Jul. 31, 1997 | Jul. 31, 1997 | FG16 | 0 | 0 |
| | Aug. 7, 1997 | Aug. 7, 1997 | FG18 | 0 | 320 |
| | Aug. 14, 1997 | Aug. 14, 1997 | FG21 | 80 | 1280+ |
| | | Aug. 28, 1997 | FG30 | 80 | 640 |
| | Sep. 11, 1997 | Sep. 12, 1997 | FG38 | | 1280++ |
| 5 | Aug. 13, 1997 | Aug. 13, 1997 | FG19 | 0 | 0 |
| | Aug. 20, 1997 | Aug. 20, 1997 | FG23 | 0 | 0 |
| | Aug. 27, 1997 | Aug. 27, 1997 | FG28 | 80 | 1280+ |
| | Sep. 3, 1997 | Sep. 3, 1997 | FG34 | 1280 | 1280+ |
| 6 | Aug. 19, 1997 | Aug. 19, 1997 | FG24 | 0 | 0 |
| | Aug. 26, 1997 | Aug. 26, 1997 | FG27 | 0 | 0 |
| | Spe. 2, 1997 | Sep. 2, 1997 | FG33 | 10 | 0 |
| | Sep. 9, 1997 | Sep. 9, 1997 | FG37 | 80 | 40 |
| 7 | Aug. 22, 1997 | Aug. 22, 1997 | FG25 | 10 | 10 |
| | Aug. 28, 1997 | Aug. 28, 1997 | FG31 | 10 | 10 |
| | Sep. 4, 1997 | Sep. 4, 1997 | FG35 | 80 | 320 |
| | Sep. 11, 1997 | Sep. 11, 1997 | FG39 | | 320 |
| 8 | Sep. 3, 1997 | Sep. 3, 1997 | FG29 | 0 | 0 |
| | Sep. 9, 1997 | Sep. 9, 1997 | FG36 | 0 | 80 |
| | Sep. 16, 1997 | Sep. 16, 1997 | FG40 | | 320 |

Legend:
Vaccination Dates: day when the fucosyl GM1-KLH vaccine was administered to the patients.
Sera Dates: day when serum from patients vaccinated with the fucosyl GM1-KLH vaccine was collected.
Sera #: Identification of serum samples collected.
ELISA results: + = at least a two-fold increase in the IgG titer; ++ = at least a four-fold increase in the IgG titer.

References for the First Three Sets of Experiments

PCT/US94/00757, filed Jan. 21, 1994, and published under WO 94/16731, published Aug. 4, 1994.

U.S. Pat. No. 4,557,931, issued on December 1985 to Irie, et al.

U.S. Pat. No. 5,102,663, issued April 1992 to Livingston, et al.

Adluri, S., et al. Immunogenicity of synthetic TF-KLH (keyhole limpet hemocyanin) and sTn-KLH conjugates in colorectal carcinoma patients. Cancer Immunol. Immunother. 41: 185-192 (1995).

Ashton, F. E., et al. Immunological properties of monoclonal antibodies to the N-propionyl derivative of group B meningococcal polysaccharide. In: Achtman, M., Kohl, P., Marchal, C., Morelli, G., Seiler, G., and Theisen, B. (eds.), Proceedings of the Seventh International Pathogenic Neisseria Conference “NEISSERIA 1990”, pp. 187-191, Berlin: Walter de Gruyter (1991).

Bernhard, H., et al. Immunorecognition of ganglioside epitopes on human normal and melanoma tissues. Int. J. Cancer 51: 568-572 (1992).

Bremer, E. G., et al. Characterization of a glycosphingolipid antigen defined by the monoclonal antibody Mbr1 expressed in normal and neoplastic epithelial cells of human mammary gland. J. Biol. Chem. 259: 14773-14777 (1984).

Brezicka, F. T., et al. Immunohistological detection of fucosyl-GM1 ganglioside in human lung cancer and normal tissues with monoclonal antibodies. Cancer Res. 49: 1300-1305 (1989).

Capurro, M., et al. FC-2.15, a monoclonal antibody active against human breast cancer, specifically recognizes Lewis X hapten. Cancer Immunol. Immunother. submitted

Chang, H. R., et al. Expression of disialogangliosides GD2 and GD3 on human soft tissue sarcomas. Cancer 70: 633-638 (1992).

Cheresh, D. A., et al. A monoclonal antibody recognizes O-acylated sialic acid in a human melanoma-associated ganglioside. J. Biol. Chem. 259: 7453-7459 (1984).

Cheung, N-K. V., et al. Ganglioside GD2 specific monoclonal antibody 3F8: a phase-I study in patients with neuroblastoma and malignant melanoma. J. Clin. Oncol. 5: 1430-1440 (1987).

Cheung, N-K. V., et al. Monoclonal antibody to a glycolipid antigen on human neuroblastoma cells. Cancer Res. 45: 2642-2649 (1985).

Dalmau J, Graus F, Cheung N-K V, Rosenblum M K, Ho A, Canete A, Delattre J-Y, Thompson S J, Posner J B. Major histocompatability proteins, Anti-Hu antibodies, and paraneoplastic encephalomyelitis in neuroblastoma and small cell lung cancer. Cancer 75: 99-109 (1995).

Dippold, W. G., et al. Immunohistochemical localization of ganglioside GD3 in human malignant melanoma, epithelial tumors, and normal tissues. Cancer Res. 45: 3699-3705 (1985).

Dippold, W. G., et al. Cell-surface antigens of human malignant melanoma: definition of six antigenic systems with monoclonal antibodies. Proc. Natl. Acad. Sci. U. S. A. 77: 6114-6118 (1980).

Dohi, T., et al. Sialylpentaosylceramide detected with anti-GM2 monoclonal antibody. J. Biol. Chem. 265: 7880-7885 (1990).

Feizi, T., Demonstration by monoclonal antibodies that carbohydrate structures of glycoproteins and glycolipids are onco-developmental antigens. Nature 314: 53-57 (1985).

Fellinger, E. J., et al. Comparison of cell surface antigen HBA71 (p30/32^{MIC2}), neuron-specific enolase, and vimentin in the immunohistochemical analysis of Ewing's sarcoma of bone. Am. J. Surg. Pathol. 16: 746-755 (1992).

Finn, O. J., Immunotherapy: tumor vaccines. Proc. Am. Assoc. Cancer Res. 36: 675 (1995).

Fredman P, Brezicka T, Holmgren J, Lindholm L, Nilsson O, Svennerholm L: Binding specificity of monoclonal antibodies to ganglioside Fuc-GM1. Biochimica et Biophysica Acta 875: 316-323 (1986).

Frosch, M., et al. NZB mouse system for production of monoclonal antibodies to weak bacterial antigens: isolation of an IgG antibody to the polysaccharide capsules of Escherichia coli K1 and group B meningococci. Proc. Natl. Acad. Sci. U. S. A. 82: 1194-1198 (1985).

Gilewski, T., et al. Preliminary results: vaccination of breast cancer patients lacking identifiable disease with MUC1-keyhole limpet hemocyanin (KLH) conjugate and QS21. Amer. Soc. Clin. Oncol. Proc. 15: 555 (1996).

Graus, F., et al. Distribution of the ganglioside GD3 in the human nervous system detected by R24 mouse monoclonal antibody. Brain Res. 324: 190-194 (1984).

Hakomori, S., Aberrant glycosylation in cancer cell membranes as focused on glycolipids: overview and perspectives. Cancer Res. 45: 2405-2414 (1985).

Hamilton, W. B., et al. Ganglioside expression on sarcoma and small cell lung cancer carcinoma compared to tumors of neuroectodermal origin (abstract) . Proc. Am. Assoc. Cancer Res. 34: 491 (1993b)

Hamilton, W. B., et al. Ganglioside expression on human malignant melanoma assessed by quantitative immune thin-layer chromatography. Int. J. Cancer 53; 566-573 (1993a).

Hayrinen, J., et al. Antibodies to polysialic acid and its N-propyl derivative: binding properties and interaction with human embryonal brain glycopeptides. J. Infect. Dis. 171: 1481-1490 (1995).

Helling F, Zhang S, Shang A, Adluri S, Calves M, Koganty R, Longenecker B M, Oettgen H F and Livingston P O. GM2-KLH conjugate vaccine: Increased immunogenicity in melanoma patients after administration with immunological adjuvant QS-21. Cancer Res 55:2783-2788 (1995).

Helling F, Shang A, Calves M, et al: GD3 vaccines for melanoma: Superior immunogenicity of keyhold limpet hemocyanin conjugate vaccines. Cancer Research 54: 197-203 (1994).

Houghton, A. N., et al. Mouse monoclonal IgG3 antibody detecting GD3 ganglioside: a phase-I trial in patients with malignant melanoma. Proc. Natl. Acad. Sci. U.S.A. 82: 1242-1246 (1985).

Hsu, S. M., et al. Use of avidin-biotin-peroxidase complex (ABC) in immuoperoxidase techniques: A comparison between ABC and unlabeled antibody (PAP) procedures. J. Histochem. Cytochem. 29: 577-580 (1981).

Husmann, M., et al. Immunohistochemical localization of polysialic acid in tissue sections: differential binding to polynucleotides and DNA of a murine IgG and a human IgM monoclonal antibody. J. Histochem. Cytochem., 38: 209-215 (1990).

Irie, R. F., and Morton, D. L., Regression of cutaneous metastatic melanoma by introlesional injection with human monoclonal antibody to ganglioside GD2. Proc. Natl. Acad. Sci. U. S. A. 83: 8694-8698 (1986).

Jones P C, Sze L L, Liu P Y, et al: Prolonged survival for melanoma patients with elevated IgM antibody to oncofetal antigen. Journal of the National Cancer Institute 66: 249-254 (1981).

Kensil C R, Patel U, Lennick M, et al: Separation and characterization of saponins with adjuvant activity from Quillaja saponaria molina cortex. Journal of Immunology 2:431-437 (1991).

Kniep B., et al. CDw60 glycolipid antigens of human leukocytes: structural characterization and cellular distribution. Blood 82: 1776-1786 (1993).

Livingston, P. O., et al. Improved survival in stage III melanoma patients with GM2 antibodies: a randomized trial of adjuvant vaccination with GM2 ganglioside. J. Clin. Oncol. 12: 1036-1044 (1994).

Livingston, P. O., Construction of cancer vaccines with carbohydrate and protein (peptide) tumor antigens. Current Opinion in Immunology 4: 624-629 (1992).

Livingston in P O, Adluri S, Helling F, Yao T-J, Kensil C R, Newman M J and Marciani D. Phase I trial of immunological adjuvant QS-21 with a GM2 ganglioside-KLH conjugate vaccine in patients with malignant melanoma. Vaccine 12:1275-1280, (1994).

Livingston P O: Approaches to augmenting the immunogenicity of melanoma gangliosides: from whole melanoma cells to ganglioside-KLH conjugate vaccines. Immunological Reviews 145:147-166 (1995).

Livingston P O, Calves M J, Natoli E J: Approaches to augmenting the immunogenicity of the ganglioside GM2 in mice: Purified GM2 is superior to whole cells Journal of Immunology 138: 1524-1529 (1987).

Livingston et al. “Characterization of IgG and IgM Antibodies Induced in Melanoma Patients by Immunization with Purified GM2 Ganglioside.” Cancer Research volume 49 (1989).

Livingston, et al. Cancer Immunology and Immunotherapy, 29: 179-184 (1989).

Macher B A, Pacuzska T, Mullin B R, et al: Isolation and identification of a fucose-containing ganglioside from bovine thyroid gland. Biochimica et biophysica Acta 588:35-43 (1979).

MacLean, G. D., et al. Active immunization of human ovarian cancer patients against a common carcinoma (Thomsen-Friedenreich) determinant using a synthetic carbohydrate antigen. J. Immunother. 11: 292-296 (1992).

MacLean, G. D., et al. Immunization of breast cancer patients using a synthetic sialyl-Tn glycoconjugate plus Detox adjuvant. Cancer Immunol. Immunother. 36: 215-222 (1993).

Menard, S., et al. Generation of monoclonal antibodies reacting with normal and cancer cells of human breast. Cancer Res. 43: 1295-1300 (1983).

Nakamura, K., et al. Chimeric anti-ganglioside GM2 antibody with antitumor activity. Cancer Res. 54: 1511-1516 (1994).

Newman M J, Wu J Y, Coughlin R T, et al: Immunogenicity and toxicity testing of an experimental HIV-1 vaccine in nonhuman primates. AIDS Research and Human Retroviruses 8:1413-1418 (1992).

Nilsson, O., et al. Detection of a ganglioside antigen associated with small cell lung carcinomas using monoclonal antibodies directed against fucosyl-GM1. Cancer Res. 46: 1403-1407 (1986)

Nishinaka, Y., et al. Development of a human monoclonal antibody to ganglioside GM2 with potential for cancer treatment. Cancer Res. 56: 5666-5671 (1996).

Ritter, et al., “Ganglioside Antigens expressed by Human Cancer Cells. ” Seminars in Cancer Biology 2: 401-409 (1991).

Saleh, M. N., et al. Phase I trial of the murine monoclonal anti-GD2 antibody 14G2a in metastatic melanoma. Cancer Res. 52: 4342-4347 (1992).

Scheidegger, E. P., et al. In vitro and in vivo growth of clonal sublines of human small cell lung carcinoma is modulated by polysialic acid of the neural cell adhesion molecule. Lab. Invest. 70: 95-106 (1994).

Schlom, J., et al. Therapeutic advantage of high-affinity anticarcinoma radioimmunoconjugates. Cancer Res. 52: 1067-1072 (1992).

Shitara, K., et al. Immunoglobulin class switch of anti-ganglioside monoclonal antibody from IgM to IgG. J. Immunol. Methods 169: 83-92 (1994).

Svennerholm, L., Chromatographic separation of human brain gangliosides. J. Neurochem. 10: 613-623 (1963).

Troy, F. A., Polysialylation: from bacteria to brains. Glycobiology 2: 5-23 (1992).

Tsuchida, T., et al. Gangliosides of human melanoma: altered expression in vivo and in vitro. Cancer Res. 47: 1278-1281 (1987b).

Tsuchida, T., et al. Gangliosides of human melanoma. J. Natl. Cancer Inst. 78: 45-54 (1987a).

Urmacher, C., et al. Tissue distribution of GD3 ganglioside detected by mouse monoclonal antibody R24. Amer. J. Dermatopathol. 11: 577-581 (1989).

Van Dessel G A F, Lagrou A R, Hilderson H J J, Dierick W S H: Structure of the major gangliosides from bovine thyroid: J Biol Chem 254: 9305-9310 (1979).

Vangsted A J, Clausen H, Kjeldsen T B, et al: Immunochemical detection of a small cell lung cancer-associated ganglioside (FucGM1) antigen in serum. Cancer Research 51: 2879-2884 (1991).

Wingo P A, Tong T, Bolden S: Cancer statistics, CA: A Cancer Journal For Clinicians 45:8-30 (1995).

Yoshino H, Ariga T, Latov N, Miyatake T, Kushi Y, Kasama T, Handa S, Yu R K: Fucosyl-GM1 in human sonsory nervous tissue is a target antigen in patients with autoimmune neuropathies. J of Neurochem 61:658-663 (1993).

Zhang, S., et al. Increased tumor cell reactivity and complement-dependent cytotoxicity with mixtures of monoclonal antibodies against different gangliosides. Cancer Immunol. Immunother. 40: 88-94 (1995).

### 4. Fourth Set of Experiments

Although SCLC is highly responsive to chemotherapy, relapses are common and most patients die within two years of diagnosis. After initial therapy, standard treatment is observation alone. Immunization against selected gangliosides as adjuvant therapy of cancer has been investigated. It has been reported that the presence of anti-GM2 ganglioside antibodies is associated with a prolonged disease-free survival in patients with melanoma, and that SCLC patients immunized with BEC2, an anti-idiotypic monoclonal antibody that mimics the ganglioside GD3, had a prolonged survival compared to historical controls. In the present trial, Fuc-GM1, another ganglioside expressed on the SCLC cell-surface, was selected as a target for active immunotherapy. Fuc-GM1 is present on most SCLC but on few normal tissues. SCLC patients achieving a major response to initial therapy were vaccinated subcutaneously on weeks 1, 2, 3, 4, 8 and 16 with Fuc-GM1 (30 μg) conjugated to the carrier protein KLH and mixed with the adjuvant QS-21. Ten patients received at least 5 vaccinations and are evaluable for response. All patients demonstrated a serologic response, with induction of both IgM and IgG antibodies against Fuc-GM1, despite prior treatment with immunosuppressive chemotherapy +/− radiation therapy. Post treatment flow cytometry demonstrated binding of antibodies from patients' sera to tumor cells expressing Fuc-GM1. In the majority of cases, sera were also capable of complement-mediated cytotoxicity. Mild transient erythema and induration at injection sites were the only consistent toxicity. The Fuc-GM1-KLH+QS-21 vaccine is safe and immunogenic in patients with SCLC. Continued study of this and other ganglioside vaccines is ongoing.

Patients and Methods

a. Patient Selection

Patients with pathologically confirmed limited or extensive stage SCLC with a documented major tumor response to therapy were eligible to participate in this study, after completion of all chemotherapy and radiation therapy which constituted part of the planned primary treatment (including prophylactic cranial irradiation, where appropriate). Patients were required to begin vaccination at least 4 weeks and no more than 12 weeks after completion of initial therapy. Eligibility criteria included Karnofsky Performance Status≧70%; age≧18; total WBC≧3.0×10⁶ cells/μl; total lymphocyte count≧0.5×⁶10 cells/μl; serum bilirubin≦1.5 mg/dl; and serum SGOT and alkaline phosphatase≦1.5×upper limit of normal. Patients with a history of seafood allergy, clinically significant peripheral neuropathy, immunodeficiency or autoimmune disease, splenectomy or splenic radiation, current use of corticosteroids, or other active malignancies within the past 5 years were excluded. All patients signed an informed consent that had been approved by the Institutional Review Board at MSKCC.

b. Vaccine Preparation and Administration

Fucosyl-GM1 was extracted and purified from bovine thyroid gland (Matreya, Inc., Pleasant Gap, Pa.). Fuc-GM1 was conjugated to KLH (Intracel Inc., Rockville, Md.) by conversion of the ceramide double bond to an aldehyde group by ozonolysis, and linked to —NH₂ groups on KLH using sodium cyanoborohydride as previously described (24). The Fuc-GM1: KLH epitope ratio was 696:1. The Fuc-GM1-KLH conjugate was washed and filtered to confirm sterility, and aliquoted into individual vials with phosphate-buffered saline. On the day of vaccination, 30 μg of the Fuc-GM1-KLH conjugate was mixed with 100 μg of QS-21 (Aquilla Biopharmaceuticals Inc., Worcester, Mass.). QS-21 is an immune adjuvant derived from a saponin fraction purified from the Quillaja saponaria Molina bark (27). The Fucosyl-GM1-KLH plus QS-21 vaccine was administered under a Food and Drug Administration Investigational New Drug Application held by MSKCC.

Patients received a series of subcutaneous vaccinations administered on weeks 1, 2, 3, 4, 8 and 16. Blood was drawn for serological testing before each vaccination, and two weeks after the fourth, fifth and sixth vaccinations. A history and physical examination, and chest x-ray was performed at week eight and eighteen. CBC, chemistries and amylase were drawn on week 3, 8, and 18. Patients were monitored for toxicity by history and physical examination, and with patient-completed diaries. The NCI common toxicity scale was used to grade toxicity except for symptoms of myalgias, fatigue, and chills, which were graded using the CALGB common toxicity scale.

Serological Assays

ELISA assays were performed to detect IgM and IgG antibody responses (25). Nunc microwell plates (Nunc, Denmark) were coated with purified Fuc-GM1 ganglioside at 0.2 μg/well in 50 μl of ethanol, and incubated at room temperature overnight. In the morning, plates were incubated with 3% HSA at 37° C. for 2 hours. Serial dilutions of patient sera were added to the plates. For IgM assays, the plates were incubated for one-hour at room temperature, washed, and then alkaline-phosphatase-conjugated goat anti-human IgM (Southern Biotechnology Assoc. Inc., Birmingham, Ala.) was added, and incubated for an additional hour at room temperature. For IgG assays, goat anti-human IgG unlabelled antibody (Southern Biotechnology Assoc. Inc., Birmingham, Ala.) was added, and incubated for one-hour. Mouse anti-goat alkaline-phosphatase-conjugated antibody (Southern Biotechnology Assoc. Inc., Birmingham, Ala.) was added and incubated for 45 minutes. Determination of IgG subclass was performed by ELISA using subclass-specific secondary mouse anti-human IgG1, IgG2, IgG3, and IgG4 monoclonal antibodies (Zymed Laboratories, Inc., San Francisco, Calif.). Alkaline-phosphatase-conjugated goat anti-mouse IgG (Southern Biotech, Birmingham, Ala.) was used as a third antibody at a dilution of 1:200.

All plates were washed and developed with Sigma 104 phosphatase substrate (Sigma Diagnostics, St. Louis, Mo.) in 10% diethanolamine. Absorbance was measured at 414 nm, and the highest dilution with an absorbance of at least 0.100 was defined as the antibody titer. To control for non-specific binding, patient sera were also tested on plates that were processed identically, but to which no ganglioside had been added, and this reading was subtracted from the value obtained in the presence of the ganglioside.

Flow cytometry assays (FACS) were performed on the human SCLC cell line H146 and the rat hepatoma cell line H4IIE, both of which express Fuc-GM1 (H4IIE more than H146). Single-cell suspensions of tumor cells (3×10⁵ cells/tube) were washed with 3% FCS in RPMI medium. Patient sera was added to the cell pellets at a 1:10 dilution, and then mixed and incubated for 30 minutes on ice. The cells were washed once with 3% FCS-RPMI and were incubated with either 20 μl of 1:25-diluted fluorescein-isothiocyanate (FITC)-labelled goat anti-human IgM (Zymed, San Francisco, Calif.) or 1:25-diluted FITC-labelled goat anti-human IgG (Southern Biotechnology Assoc. Inc., Birmingham, Ala.) on ice for 30 minutes. The percent-positive cell population and the mean fluorescence intensity (MFI) of the stained cells were analyzed by flow cytometry (FACScan, Becton-Dickinson, Calif.). The mouse anti-Fuc-GM1 monoclonal antibody, F12, was used as a positive control, and pretreatment sera were used as negative controls. FACS inhibition studies were performed on select sera using Fuc-GM1 antigen, and GD3 ganglioside as a negative control.

Complement-dependent cytotoxicity assays (CDC) were performed by a 2-hour chromium release assay. The Fuc-GM1-positive cell lines H146 and H4IIE served as target cells. Approximately 10⁷ cells were labeled with 100 μCi of Na₂⁵¹CrO₄ (New England Nuclear, Boston, Mass.) in 3% HSA for 2 hours at 37° C., shaking every 15 minutes. The cells were washed four times, and brought to a concentration of 10⁶ live cells/ml. Fifty microliters of labeled cells were added to 50 μl of diluted (1:2) pre or postvaccination serum or with medium alone in 96-well round-bottomed plates (Corning, New York), and incubated at 40° C. on a shaker for 45 minutes. Human complement diluted 1:5 with 3% HSA was added, at 100 μl/well, and incubated at 37° C. for 2 hours. The plates were spun at 100 g for 5 minutes, and an aliquot of 100 μl of supernatant from each well was read by a gammacounter to determine the amount of ⁵¹Cr released. All samples were performed in triplicate and included control wells for maximum release and for spontaneous release in the absence of complement. Spontaneous release (the amount released by target cells incubated with complement alone) was subtracted from both experimental and maximal release values. Maximum release was the amount released by target cells after a 2-hour incubation with 20 μl of 10% triton X-100 (Sigma Diagnostics, St. Louis, Mo.) and 100 μl of human complement. Specific release was equal to corrected experimental release divided by corrected maximal release:

$$Specific release ( \% ) = \frac{experimental release - spontaneous release}{maximum release - spontaneous release} \times 100$$

Patients were considered evaluable for immunologic response if they had received at least five vaccinations. Sera were considered positive by ELISA if the titer of reactivity was at least 1:40, by FACS if the percent-positive difference between the pre and post vaccination sera was at least 20%, and by CDC if there was 10% or more specific release.

Results

a. Patient Characteristics

Thirteen patients received the Fuc-GM1-KLH conjugate plus QS-21 vaccine. Patient characteristics are listed in Table 5. Nine patients had extensive stage SCLC, and 4 patients had limited stage disease. The median age was 52 years (range 43-76 years), with a median KPS of 90%. All patients received a platinum-based chemotherapy regimen, six patients received thoracic radiation, and 4 patients were treated with prophylactic cranial radiation as part of the initial planned therapy. Of the 13 patients on study, three patients relapsed prior to receiving the fifth vaccination, and therefore only 10 patients are evaluable for serologic response. Of these ten patients, four received only 5 vaccinations secondary to relapsed SCLC prior to completing the protocol therapy. At the time of disease progression, patients were taken off study, and further therapy was at the discretion of the patient's physician.

Serologic Assays

All ten evaluable patients demonstrated an antibody response by ELISA to the Fuc-GM1-KLH conjugate vaccine, with high titers of both IgM and IgG antibodies against Fuc-GM1 despite prior treatment with immunosuppressive chemotherapy with or without radiation therapy (Table 6). IgG antibodies were primarily of the IgG1 subclass (Table 7 (some patients with lower IgG titers than those listed in Table 6)). Of the three evaluable patients with limited stage disease, each received all of the 6 planned vaccinations, with induction of IgM and IgG antibody titers of 1:320-1:2560 and 1:1280-1:2560, respectively. Although 4 patients with extensive stage disease relapsed prior to receiving the sixth vaccination, the majority of these patients demonstrated both an IgM and IgG response. The interval between diagnosis of SCLC and first immunization did not appear to affect the antibody titers.

FACS analysis using the Fuc-GM1-positive rat hepatoma cell line H4IIE demonstrated post-treatment IgM and IgG antibodies from 8 of 10 patients and 5 of 10 patients, respectively, that bound to tumor cells (Table 8). Results using the human SCLC cell line H146, which expresses lower levels of Fuc-GM1 than H4IIE, showed post-treatment IgM and IgG antibodies that bound to tumor cells from 5 of 10 patients and 1 of 10 patients, respectively (Table 8). Addition of the Fuc-GM1 antigen to selected patient sera inhibited subsequent binding of antibody to tumor cells, whereas binding to tumor cells was not inhibited by the addition of GD3. Post-vaccination sera from 8 of 10 patients evaluable for response induced complement-mediated cytotoxicity of the Fuc-GM1-positive tumor cell lines (Table 9).

Toxicity

All 13 patients were evaluable for toxicity, and toxicity data from patient-completed diaries was available for 61 of the 68 administered vaccinations. The most common toxicity was a local skin reaction, which occurred after the majority of vaccinations and typically consisted of mild pain, swelling and erythema at the injection site. This reaction lasted approximately 2-5 days, and was most pronounced after the second or third vaccination. Mild, transient flu-like symptoms including low-grade fever, myalgias, headache, and chills occurred after a minority of vaccinations. Diarrhea was observed after 9 of 60 (15%) vaccinations (8 grade 1, 1 grade 2). Grade 1 fatigue was observed after 13 of 60 (22%) vaccinations. One patient developed pneumonia associated with chest pain and shortness of breath after the first vaccination. This responded to antibiotics, and was not felt to be treatment related. Worsening of chemotherapy-induced sensory neuropathy by one toxicity grade was observed in 6 patients (Table 10). Three patients had worsening of sensory neuropathy from grade 0 to grade 1, 2 patients from grade 1 to grade 2, and one patient from grade 2 to grade 3. No motor neuropathy was observed.

Discussion

Fuc-GM1 is extensively expressed on most small cell lung cancers and minimally expressed on normal tissues, suggesting that this ganglioside antigen may serve as an excellent target for active immunization. However, the immunogenicity of auto-antigens such as Fuc-GM1 can not be consistently predicted based on expression in normal cells. The GD3 ganglioside, which has a restricted distribution on normal cells that is limited to the brain, connective tissue and a small population of T-cells, has proven to be poorly immunogenic in humans (28, 29), although occasional antibody responses against GD3 have been induced (30). In contrast, GM2, which is expressed in the brain as well as the secretory borders of all epithelial tissues, has proven to be highly immunogenic. Fuc-GM1 has a more restricted distribution on normal tissues than either GM2 or GD3, and therefore would be expected to be more immunogenic. This study demonstrates that indeed this is the case.

Mean peak ELISA antibody titers against Fuc-GM1 after immunization with the Fuc-GM1-KLH plus QS-21 vaccine were 1:320 for IgM and 1:960 for IgG. These titers are similar to the titers induced against GM2 with the GM2-KLH plus QS-21 vaccine in previous trials in melanoma patients (25, 26), but the melanoma patients were free of detectable disease and had not received previous chemotherapy or radiation therapy. The majority of the SCLC patients treated in this trial had recently completed treatment with chemotherapy with or without radiation therapy and continued to have radiologic evidence of evaluable disease. Despite the greater extent of disease and prior therapy, the Fuc-GM1-KLH conjugate vaccine consistently induced IgM and IgG antibody responses.

Based on these results, Fuc-GM1 appears to be the most immunogenic of the gangliosides we have tested, clearly more immunogenic than GD2 and GD3, and at least as immunogenic as GM2. Using the rat hepatoma cell line H4IIE, with extensive cell surface expression of Fuc-GM1, and the SCLC cell line H146, with more modest cell surface expression of Fuc-GM1, reactivity of the sera was demonstrated by both flow cytometry and CDC. Eight of 10 patients and 6 of 10 patients showed at least a doubling of the percent-cells bound by IgM and IgG flow cytometry against H4IIE, respectively. Specificity of these reactions for cell surface Fuc-GM1 was demonstrated in selected cases by complete inhibition of all reactivity of post immunization sera following the addition of purified Fuc-GM1, but not GD3, to the reactions. At least a doubling of CDC against H4IIE was seen in 6 of 10 patients, and two-thirds of the patients had at least 70% cytotoxicity induced by post vaccination sera. Increases were seen against both H146 and H4IIE, but were more significant against H4IIE. The IgG antibodies were primarily of the IgG1 subclass, so it is likely that this CDC was induced by both IgM and IgG antibodies, and that the two were additive as we have demonstrated previously for antibodies against GM2 (30).

The Fucosyl-GM1-KLH+QS-21 vaccine was generally well tolerated. Mild transient erythema and induration at the injection sites were observed in most patients, associated with occasional flu-like symptoms. Slight increases in the severity of sensory neuropathy (by one NCI toxicity grade) were observed in 6 patients during the course of the study. This was in the setting of an objective but not clinically significant baseline neuropathy in 8 of 13 patients (62%) entering the study, presumably related to prior chemotherapy. Binding of antibody to Fuc-GM1 expressed on peripheral sensory neurons is a possible explanation for the observed changes, however the changes were mild, with the majority of patients reporting no change in functional capacity or progressive worsening of symptoms over time. There was no evidence for diabetes mellitus, gastrointestinal or immunologic dysfunction, or other problems to suggest potential autoimmunity based on Fuc-GM1 distribution on normal tissues.

It has been demonstrated here that 1) despite the recent intensive chemotherapy, these SCLC patients exhibited a good antibody response against this conjugate vaccine, 2) Fuc-GM1 is highly immunogenic, and 3) immunization with Fuc-GM1 is not associated with significant toxicity. However, while Fucosyl GM1 is expressed on most SCLC cells in most specimens, it is not expressed in every cell or in every specimen. To effectively target every SCLC cell, a polyvalent vaccine containing multiple consistently immunogenic antigens will be required. This was an initial trial with a KLH conjugate plus QS21 vaccine in patients with SCLC, but there has been considerable experience with such conjugate vaccines in patients with other cancers. Of the cell surface antigens known to be expressed in the majority of SCLCs, consistently immunogenic vaccines against GM2, GD2, Globo H and now Fucosyl GM1 are available (25, 26, 31, 32, 33). Trials with a KSA-KLH conjugate vaccine are in progress in patients with other cancers, and we have begun to immunize SCLC patients with a polysialic acid-KLH conjugate vaccine. GD3 and 9-0-acetyl GD3 vaccines have not resulted in consistent demonstrable antibodies against these antigens (28, 29), and therefore are not considered good candidates for inclusion in the polyvalent vaccine, although trials with GD3-KLH and BEC2 vaccines administered to the same group of patients are planned for the imminent future.

Consequently, of the identified potential targets for antibody mediated immunotherapy of SCLC, vaccines against four antigens are now available and immunogenicity of the final two or three antigens will be determined by the end of 1998. At that time the availability of a consistently immunogenic polyvalent vaccine containing 4-6 immunogenic antigens should permit us to determine conclusively for the first time the potential for antibody inducing vaccines which target essentially every SCLC cell in every patient's tumor.

TABLE 5
Patient Characteristics

| | # PATIENTS |
|---|---|
| Number of patients on study | 13 |
| Median age, years (range) | 52 (43-76) |
| Median Karnofsky Performance Status (range) | 90% (70-100%) |
| Male/Female | 5/8 |
| Patients evaluable for serologic response | 10 |
| Stage of disease | |
| Limited | 4 |
| Extensive | 9 |
| Prior Therapy | |
| Chemotherapy | 13 |
| Radiation therapy | |
| Chest irradiation | 6 |
| Prophylactic cranial irradiation | 4 |

TABLE 6
ELISA Titers Against the Fuc-GM1 Antigen Pre and Peak Post
Vaccination for the 10 Patients Evaluable for Response

| | | | Time since | ELISA | | | |
|---|---|---|---|---|---|---|---|
| | | Number of | diagnosis to | Reciprocal Titers | | | |
| Patient | Stage of | vaccinations | vaccination | IgM | | IgG | |
| number | SCLC | received | (months) | pre | peak | pre | peak |
| 1 | Extensive | 5 | 9 | 10 | 320 | 0 | 320 |
| 2 | Extensive | 6 | 5 | 0 | 640 | 0 | 640 |
| 3* | Limited | 6 | 16 | 0 | 2560 | 0 | 2560 |
| 4 | Extensive | 6 | 7 | 0 | 1280 | 10 | 2560 |
| 5 | Extensive | 5 | 5 | 0 | 320 | 0 | 40 |
| 6 | Limited | 6 | 6 | 0 | 320 | 0 | 1280 |
| 7 | Extensive | 6 | 6 | 10 | 320 | 10 | 320 |
| 8 | Limited | 6 | 8 | 0 | 320 | 0 | 1280 |
| 9 | Extensive | 5 | 5 | 0 | 1280 | 0 | 1280 |
| 10 | Extensive | 5 | 6 | 0 | 320 | 0 | 40 |

*treated with permission from our Institutional Review Board, as more than 12 weeks had elapsed since completion of initial therapy and the start of vaccination

TABLE 7
Peak Post Vaccination ELISA Titers for IgG Subclass Analysis

| | |
|---|---|
| Patient | ELISA Reciprocal Subclass Titers |

| number | IgG | IgG1 | IgG2 | IgG3 | IgG4 |
|---|---|---|---|---|---|
| 1 | 320 | 40 | 0 | 0 | 20 |
| 2 | 640 | 640 | 0 | 80 | 0 |
| 3 | 640 | 160 | 10 | 20 | 80 |
| 4 | 1280 | 1280 | 0 | 10 | 80 |
| 5 | 40 | 0 | 0 | 0 | 0 |
| 6 | 640 | 640 | 10 | 20 | 80 |
| 7 | 320 | 0 | 0 | 0 | 0 |
| 8 | 0 | 0 | 0 | 0 | 0 |
| 9 | 1280 | 640 | 0 | 0 | 0 |
| 10 | 20 | 10 | 10 | 0 | 10 |

TABLE 8
Flow Cytometry Assays (FACS)
Results using the rat hepatoma cell line H4IIE and the human SCLC cell
line H146 pre and post vaccination (% positive cells)

| H4IIE | | H146 | |
|---|---|---|---|
| IgM | IgG | IgM | IgG |

| | | |
|---|---|---|
| Patient | % positive cells | % positive cells |

| | | | | | | | | |
|---|---|---|---|---|---|---|---|---|
| number | pre | post | pre | post | pre | post | 0pre | post |

| | | | | | | | | |
|---|---|---|---|---|---|---|---|---|
| 1 | 10.0 | 30.2 | 11.2 | 28.2 | 10.9 | 52.3 | 9.9 | 21.4 |
| 2 | 9.6 | 14.4 | 10.8 | 14.7 | 11.1 | 16.8 | 10.7 | 26.6 |
| 3 | 10.8 | 56.5 | 10.7 | 46.4 | 9.9 | 43.3 | 10.7 | 35.6 |
| 4 | 11.3 | 51.2 | 9.9 | 41.6 | 10.0 | 11.9 | 10.4 | 10.3 |
| 5 | 10.0 | 57.2 | 9.9 | 39.5 | 11.2 | 23.5 | 10.0 | 10.7 |
| 6 | 9.9 | 84.4 | 9.5 | 47.6 | 10.9 | 21.7 | 10.7 | 18.4 |
| 7 | 10.5 | 87.1 | 11.0 | 47.4 | 10.2 | 98.4 | 10.6 | 17.8 |
| 8 | 12.0 | 85.9 | 9.3 | 3.2 | 10.1 | 57.7 | 10.6 | 1.8 |
| 9 | 8.3 | 97.7 | 9.1 | 15.6 | 10.8 | 51.5 | 10.0 | 16.0 |
| 10 | 92.6 ? | 88.9 ? | 5.1 | 3.92 | 57.2 | 63.4 | 7.8 | 6.6 |
| positive- | | | | | | | | |
| control* | | | | | | | | |

*Anti-Fuc-GM1 mouse monoclonal antibody

TABLE 9
Complement-Dependent Cytotoxicity Assays (CDC)
Results using the rat hepatoma cell line H4IIE and the human SCLC cell
line H146 pre and post vaccination (% lysis)

| | H 4IIE | | H 146 | |
|---|---|---|---|---|
| Patient | % lysis | | % lysis | |
| number | pre | post | pre | post |
| 1 | 9 | 104 | 41 | 97 |
| 2 | 29 | 100 | 34 | 58 |
| 3 | 27 | 89 | 27 | 47 |
| 4 | 59 | 100 | 37 | 49 |
| 5 | 42 | 51 | 0 | 0 |
| 6 | 20 | 73 | 0 | 23 |
| 7 | 53 | 45 | 4 | 23 |
| 8 | 8 | 86 | 0 | 0 |
| 9 | 36 | 71 | 33 | 64 |
| 10 | 3 | 59 ? | 0 | 11 |
| Control* | | 100 | | 18 |

*Anti-Fuc-GM1 mouse monoclonal antibody, F12

TABLE 10
Neurotoxicity
Sensory Neuropathy - For 13 patients who have completed the vaccine
protocol or stopped treatment early due to relapse

| | | Highest Grade | | |
|---|---|---|---|---|
| Pretreatment | | During Trial | | |
| Grade (# pts.) | 0 | 1 | 2 | 3 |
| Grade 0 (5) | 2 | 3 | 0 | 0 |
| Grade 1 (4) | 0 | 2 | 2* | 0 |
| Grade 2 (4) | 0 | 0 | 3 | 1 |

Shaded area represents number of patients who had an increase in sensory neuropathy by one grade during the course of the trial.
*One patient had a transient increase to grade 2 sensory neuropathy that subsequently improved to grade 1 (baseline).

References for the Fourth Series of Experiments

1. Landis, S. H., Murray T., Bolden, S., and Wingo, P. A. Cancer Statistics. CA Cancer J Clin., 48: 6-29, 1998.

2. Ihde, D. C. Small cell lung cancer: State of the art therapy 1994. Chest, 107: 243S-248S, 1995.

3. Hamilton, W. B., Helling, F., and Livingston, P. O. Ganglioside expression on sarcoma and small cell lung carcinoma compared to tumors of neuroectodermal origin. Proc Am Assoc Cancer Res., 34: 491, 1993.

4. Zhang, S., Cordon-Cardo, C., Zhang, H. S., Reuter, V. E., Adluri, S., Hamilton, W. B., Lloyd, K. O., and Livingston, P. O. Selection of tumor antigens as targets for immune attack using immunohistochemistry. I. Focus on gangliosides. Int. J. Cancer, 73: 42-49, 1997.

5. Brezicka, F-T., Olling, S., Nilsson, O., Bergh, J., Holmgren, J., Sörenson, S., Yngvason, F., and Lindholm, L. Immunohistological detection of fucosyl-GM1 ganglioside in human lung cancer and normal tissues with monoclonal antibodies. Cancer Res., 49: 1300-1305, 1989.

0. Fuentes, R., Allman, R., and Mason, M. D. Ganglioside expression in lung cancer cell lines. Lung Cancer, 18: 21-33, 1997.

7. Brezicka, F-T., Olling, S., Bergman, B., Berggren, H., Engström, C-P., Hammarström, S., Holmgren, J., Larsson, S., and Lindholm, L. Coexpression of ganglioside antigen Fuc-GM1, neural-cell adhesion molecule, carcinoembryonic antigen, and carbohydrate tumor-associated antigen CA 50 in lung cancer. Tumor Biol, 13: 308-315, 1992.

8. Cheresh, D. A., Rosenberg, J., Mujoo, K., Hirschowitz, L., and Reisfeld, R. A. Biosynthesis and expression of the disialoganglioside GD2, a relevant target antigen on small cell lung carcinoma for monoclonal antibody-mediated cytolysis. Cancer Res., 46: 5112-5118, 1986.

9. Grant S. C., Kostakoglu, L., Kris, M. G., Yeh, S. D. J., Larson, S. M., Finn, R. D., Oettgen, H. and Cheung, N-K. V. Targeting of small cell lung cancer using the anti-GD2 ganglioside monoclonal antibody 3f8: a pilot trial. European Journal of Nuclear Medicine, 23: 145-149, 1996.

10. Zhang, S., Zhang, H. S., Cordon-Cardo, C., Ragupathi, G. and Livingston, P. O. Selection of tumor antigens as targets for immune attack using immunohistochemistry: III. Protein antigens. Clin. Cancer Res., in press.

11. Cheung, N-K. V., Lazarus, H., Miraldi, F. D., Abramowsky, C. R., Kallick, S., Saarinen, U. M., Spitzer, T., Strandjord, S. E., Coccia, P. F., and Berger, N. A. Ganglioside GD2 specific monoclonal antibody 3F8: a phase I study in patients with neuroblastoma and malignant melanoma. J Clin Oncol., 5: 1430-1440, 1987.

12. Houghton, A. N., Mintzer, D., Cordon-Cardo, C., Welt, S., Fliegel, B., Vadhan, S., Carswell, E., Melamed M. R., Oettgen, H. F., and Old, L. J. Mouse monoclonal IgG3 antibody detecting GD3 ganglioside: a phase I trial in patients with malignant melanoma. Proc Natl Acad Sci., 82: 1242-1246, 1985.

13. F. Irie, R. , and Morton, D. L. Regression of cutaneous metastatic melanoma by intralesional injection with human monoclonal antibody to ganglioside GD2. Proc Natl Acad Sci., 83: 8694-8698, 1986.

14. Irie, R. F., Matsuki, T., and Morton, D. L. Human monoclonal antibody to ganglioside GM2 for melanoma treatment [letter]. Lancet 8641: 786, 1989.

15. Jones, P. C., Sze, L. L., Liu, P. Y., Morton, D. L., and Irie, R. F. Prolonged survival for melanoma patients with elevated IgM antibody to oncofetal antigen. JNCI 66: 249-254, 1981.

16. Livingston, P. O., Wong, G. Y. C., Adluri, S., Tao Y., Padavan, M., Parente, R., Hanlon C., Calves, M. J., Helling, F., Ritter, G., Oettgen, H. F., and Old, L. J. Improved survival in Stage III melanoma patients with GM2 antibodies: a randomized trial of adjuvant vaccination with GM2 ganglioside. J. Clin Oncol., 12: 1036-1044, 1994.

17. Livingston, P. O., Ritter, G., Srivastava, P., Padavan, M., Calves, M. J., Oettgen, H. F., and Old, L. J. Characterization of IgG and IgM antibodies induced in melanoma patients by immunization with purified GM2 ganglioside. Cancer Res., 49: 7045-7050, 1989.

18. Grant, S. C., Yao, T. J., Kris, M. G., Rigas, J. R., Pisters, K. M. W., Miller, V., Houghton, A. H., and Chapman, P. B. Long survival following immunization with BEC2 plus BCG after initial therapy for small cell lung cancer. Proc Am Soc Clin Oncol., 15: 555A, 1996.

19. Macher, B. A., Pacuszka, T., Mullin, B. R., Sweeley, C. C., Brady, R. O., and Fishman, P. H. Isolation and identification of a fucose-containing ganglioside from bovine thyroid gland. Biochimica et Biophysica Acta, 588: 35-43, 1979.

20. Fredman, P., Brezicka, T., Holmgren, J., Lindholm, L., Nilsson, O., and Svennerholm, L. Binding specificity of monoclonal antibodies to ganglioside, Fuc-GM1. Biochimica et Biophysica Acta, 875: 316-323, 1986.

21. Vangsted, A. J., Clausen, H., Kjeldsen, T. B., White, T., Sweeney, B., Hakomori, S., Drivsholm, L., and Zeuthen, J. Immunochemical detection of a small cell lung cancer-associated ganglioside (Fuc-GM1) antigen in serum. Cancer Res., 51: 2879-2884, 1991.

22. Yoshino, H., Ariga, T., Latov, N., Miyatake, T., Kushi, Y., Kasama, T., Handa, S., Yu, R. K. Fucosyl-GM1 in human sensory nervous tissue is a target antigen in patients with autoimmune neuropathies. J Neurochem 61: 658-663, 1993.

23. Livingston, P. O., Natoli, E. J., Calves, M. J., Stockert, E., Oettgen, H. F., and Old, L. J. Vaccines containing purified GM2 ganglioside elicit GM2 antibodies in melanoma patients. Proc Natl Acad Sci., 84: 2911-2915, 1987.

24. Helling, F., Shang, A., Calves, M., Zhang, S., Ren, S., Yu, R. K., Oettgen, H. F., and Livingston, P. O. GD3 vaccines for melanoma: superior immunogenicity of keyhole limpet hemocyanin conjugate vaccines. Cancer Res., 54: 197-203, 1994.

25. Helling, F., Zhang, S., Shang, A., Adluri, S., Calves, M., Koganty, R., Longenecker, B. M., Yao, T. J., Oettgen H. F., and Livingston, P. O. GM2-KLH conjugate vaccine: increased immunogenicity in melanoma patients after administration with immunological adjuvant QS-21. Cancer Res., 55: 2783-2788,1995.

26. Livingston, P. O., Adluri, S., Helling, F., Yao, T. J., Kensil, C. R., Newman, M. J., and Marciani, D. Phase I trial of immunological adjuvant QS-21 with a GM2 ganglioside-keyhole limpet hemocyanin conjugate vaccine in patients with malignant melanoma. Vaccine 12: 1275-1280, 1994.

27. Kensil, C. R., Patel, U., Lennick, M., and Marciani, D. Separation and characterization of saponins with adjuvant activity from Quillaja saponaria molina cortex. J Immunology, 146: 431-437, 1991.

28. Ritter, G., Boosfeld, E., Adluri, R., Calves, M., Ren, S., Yu, R. K., Oettgen, H. F., Old, L. J. and Livingston, P. O. Analysis of the antibody response to immunization with the purified O-acetyl GD3 gangliosides in patients with malignant melanoma. Int. J. Cancer. 62: 1-5, 1995.

29. Ritter, G., Boosfeld, E., Adluri, R., Calves, M., Oettgen, H. F., Old, L. J. and Livingston, P. O. Antibody response to immunization with the ganglioside GD3 and GD3 congeners (lactones, amide and gangliosidol) in patients with malignant melanoma. Int. J. Cancer, 48: 379-385, 1991.

30. Ragupathi, G., Meyers, M., Adluri, S., Ritter, G., and Livingston, P. O. Phase I trial with GD3-lactone-KLH conjugate and immunological adjuvant QS-21 vaccine with malignant melanoma. Proc Am Assoc Cancer Res., 38: 398, 1997.

31. Livingston, P. O., Zhang, S., Walberg, L., Ragupathi, G., Helling, F., and Fleischer, M. Tumor cell reactivity mediated by IgM antibodies in sera from melanoma patients vaccinated with GM2-KLH is increased by IgG antibodies. Ca Immunol. and Immunotherapy 43: 324-330, 1997.

32. Livingston, P. O. Approaches to augmenting the is immunogenicity of melanoma gangliosides: From whole melanoma cells to ganglioside-KLH conjugate vaccines. Immunol. Reviews 145: 147-166, 1995.

33. Ragupathi, G., Park, T. K., Zhang, S., Kim, I. J., Graeber, K., Adluri, S., Lloyd, K. O., Danishefsky, S. J., and Livingston, P. O. Immunization of mice with the synthetic hexasaccharide Globo H results in antibodies against human cancer cells. Angewandte Chemie 36: 125-128, 1997.

What is claimed is:

1. A composition comprising a fucosyl GM1 ganglioside or a oligosaccharide portion thereof conjugated to an immunogenic protein, an adjuvant, the amounts of such conjugated ganglioside and such adjuvant being effective to stimulate or enhance antibody production in a subject, and a pharmaceutically acceptable carrier.

2. The composition of claim 1, wherein the composition comprises fucosyl GM1 ganglioside conjugated to Keyhole Limpet Hemocyanin or a derivative thereof conjugated to the ganglioside through the ceramide portion thereof.

3. The composition of claim 2, wherein the ganglioside is conjugated to Keyhole Limpet Hemocyanin or derivative thereof.

4. The composition of claim 1, wherein the adjuvant is a carbohydrate derived from the bark of a Quillaja saponaria Molina tree.

5. The composition of claim 4, wherein the carbohydrate is QS-21.

6. The composition of claim 1, wherein the amount of the ganglioside is an amount between about 3 μg to about 100 μg.

7. The composition of claim 5, wherein the amount of QS-21 is an amount between about 30 μg to about 100 μg.

8. The composition of claim 1, wherein the subject is a human.

9. A method of stimulating antibody production in a subject which comprises administering to the subject an effective amount of the composition of claim 1 to stimulate antibody production.

10. The method of claim 9, wherein the composition comprises fucosyl GM1 ganglioside conjugated to Keyhole Limpet Hemocyanin or a derivative thereof conjugated to the ganglioside through the ceramide portion thereof.

11. A method of enhancing antibody production in a subject which comprises administering to the subject an effective amount of the composition of claim 1 to enhance antibody production.

12. A method of preventing cancer in a subject which comprises administering to the subject an amount of the composition of claim 1 effective to prevent cancer.

13. The method of claim 12, wherein the cancer is small cell lung cancer.

14. A method of treating cancer in a subject which comprises administering to the subject an amount of the composition of claim 1 effective to treat cancer.

15. The method of claim 14, wherein the cancer is small cell lung cancer.
